(19) United States
(12) Patent Application Publication
(10) Pub. No.: US 2019/0151575 A1
(43) Pub. Date: May 23, 2019

(54) RESPIRATORY UNIT WITH MULTIPLE MEDICATION CHAMBERS

(71) Applicant: Ali Wahab A. ALFARAJ, Oakville (CA)

(72) Inventor: Ali Wahab A. ALFARAJ, Oakville (CA)

(21) Appl. No.: 15/816,885

(22) Filed: Nov. 17, 2017

(51) Int. Cl. A61M 15/00; A61M 11/00; A61M 16/10; A61M 16/16

(52) U.S. Cl. CPC A61M 15/0003; A61M 15/0085; A61M 15/0066; A61M 15/0086; A61M 11/007; A61M 11/005; A61B 5/14552; A61M 16/16; A61M 2205/18; A61M 2205/3303; A61M 2205/3553; A61M 2205/36; A61M 2205/502; A61M 16/109

(57) ABSTRACT

Disclosed are respiratory devices comprising at least four components selected from a control unit 102, a filling unit 104, a generation and output unit 106, and an input unit 108, wherein the respiratory device comprises a nebulizer mode and a steam mode of operation. Also disclosed are plungers 402, comprising a shaft 404; a handle 406; and a plunger joint 414 at the plunger distal end 412, wherein the plunger joint 414 connects securely with a seal joint 410 of a moveable seal 408 of an administrable material chamber 302,304. The plunger is used to fill administered material chambers 302,304.

### FIELD OF THE INVENTION

The present invention is in the field of medical devices. Specifically, the present invention is in the field of respiratory aid units for use at hospitals, home or other non-hospital settings.

### BACKGROUND OF THE DISCLOSURE

Acute and chronic respiratory mortality and morbidity are significant, common and prevalent health problems that affect patients' quality of life, and are a big burden on the public health system. Recently, the incidents of respiratory diseases have been increasing. In some countries, the prevalence of bronchial asthma alone can reach up to 21%.

A variety of respiratory units are used for the treatment of obstructive airway diseases, such as bronchial asthma, chronic obstructive pulmonary disease (COPD), cystic fibrosis, viral and bacterial pneumonia, bronchiolitis, emphysema, hyperactive airway disease, wheezing attacks (e.g., as associated with seasonal flu or allergies) and others.

Inhaled medications, such as salbutamol, corticosteroids and ipratropium bromide, are very effective in the treatment of a wide spectrum of acute and chronic respiratory conditions. In addition, in some cases such as croup (common viral upper respiratory infection), inhalation of water steam alone is the main core of treatment. In some cases, a patient greatly benefits from receiving a cocktail of medications as opposed to just a single one. Current respiratory units do not allow for multiple medications to be administered by inhalation simultaneously.

It is not uncommon for a patient suffering from a respiratory disease to require rapid adjustment to medications due to an acute onset of severe symptoms. Given the current state of the art, it is nearly impossible for patients to reach their doctor in time. Instead, these patients end up at emergency rooms, causing significant economic damage due to their own loss of productivity and the additional burden on the emergency rooms.

Therefore, a need exists in the art for a respiratory unit that can administer multiple medications simultaneously, and can be in communication with the patient's health care provider.

### SUMMARY OF THE INVENTION

Disclosed are respiratory devices comprising at least four components selected from a control unit 102, a filling unit 104, a generation and output unit 106, and an input unit 108, wherein the respiratory device comprises a nebulizer mode and a steam mode of operation. Also disclosed are plungers 402, comprising a shaft 404; a handle 406; and a plunger joint 414 at the plunger distal end 412, wherein the plunger joint 414 connects securely with a seal joint 410 of a moveable seal 408 of an administrable material chamber 302,304. The plunger is used to fill administrable material chambers 302,304.

### BRIEF DESCRIPTION OF THE DRAWINGS

FIG. 1 illustrates an embodiment of the disclosed respiratory devices.

FIG. 2 illustrates an embodiment of the disclosed control unit of the disclosed respiratory devices.

FIG. 3 illustrates an embodiment of the disclosed filling unit of the disclosed respiratory devices.

FIG. 4 illustrates an embodiment of the disclosed filling mechanism.

FIG. 5 illustrates an embodiment of the disclosed water tank.

FIG. 6 illustrates an embodiment of the disclosed generator unit.

FIG. 7 illustrates an embodiment of the disclosed internal design.

FIG. 8 illustrates an embodiment of the disclosed output stick.

### DETAILED DESCRIPTION OF THE EMBODIMENTS

### Parts List

The following list of parts refers to the accompanying drawings:
100: An embodiment of the disclosed device.
102: Control unit.
104: Filling unit.
106: Generation and output unit.
108: Input unit.
110: Output stick.
202: Power button.
204: Display monitor.
206: Chamber lights.
208,210: Navigation buttons.
212: Nebulization rate indicator.
214: Bluetooth® light indicator.
216: Nebulization mode display light.
218: Steam mode display light.
220: Speaker.
302: Medication chambers.
304: Carrier chamber.
306: Chamber housing walls.
308: Chamber outlet tube.
310: Opening from the chamber to the conduit.
312: Housing for medication or carrier chamber.
402: Plunger.
404: Plunger shaft.
406: Plunger handle.
408: Chamber moveable seal.
410: Seal joint.
412: Plunger distal end.
414: Plunger joint.
416: Upper end of the chamber.
418: Chamber fill lines.
502: Water tank.
504: Water tank filling cap.
506: Conduit connecting the water tank to the device.
508: Conduit valve.
510: Connector to secure the water tank to the device.
512: Lower end of the device.
514: Upper end of the device.
602: Ultrasound generator.
604: Water sink.
606: Medication collector.
702: Air pressure pump.
704: Air pressure line.
706: Air inlet for medication or carrier chamber.
708: Conduit connecting medication or carrier chamber to the medication collector.
710: Air pressure valve.
802: Proximal section of output stick.
804: Distal section of output stick.
806: Connector.

Disclosed herein are ultrasonic nebulizer and steam generator respiratory devices having a plurality of chambers for medication. The disclosed respiratory devices send real time physiological data of the user to a health care professional (HCP) via the internet. Based on the data, the HCP can then submit an order to manipulate the dosage of the various medications stored in the respiratory device. The respiratory device will then implement the order, e.g., change the medication dosage, once the device obtains the user's approval, or will issue various alerts.

The respiratory devices disclosed herein generally comprise at least four components that work in concert to achieve the end result. These components include a control unit, a filling unit, a generator and output unit, and an input or accessories unit.

The control unit comprises the electronic panel and switches that allow for the function of the device, such as, without limitation, mode selector (nebulizer or steam generator), dose selectors, timers, and any message or alert displays. The filling unit comprises the plurality of chambers or reservoirs for the various medications, water, or any other compound that is to be administered nasally (e.g., by inhalation) to the patient. The generator and output unit nebulizes the medications or generates steam for their administration, as is necessary. Finally, the input or accessories unit provides the means for a water tank connection, wired or wireless electronic communications with between the disclosed respiratory devices and other.

In some embodiments, the disclosed respiratory devices are configured to communicate with one or more devices that obtain physiological data on the user of the device. The data includes, but is not limited to, respiratory rate, peak expiratory respiratory volume, pulse rate, blood oxygen saturation limits, blood pressure, body temperature, and the like. In some embodiments, the disclosed respiratory devices comprise sensors for measuring the physiological data. In other embodiments, the data is obtained by separate instruments and are relayed to the disclosed respiratory devices either by wired or wireless communication.

In certain embodiments, the disclosed respiratory devices communicate the data to an HCP via the internet. In some embodiments, the data is relayed continuously while in other embodiments, the data is relayed at certain time intervals. In certain embodiments, the HCP sets up certain threshold maxima and minima for the data. Once the measured physiological function passes a threshold, the device sends and urgent alert to the HCP to review the data immediately. In some of these embodiments, when the threshold value is passed, the disclosed respiratory devices emit and audible signal to alert someone in the user's vicinity that immediate action is to be taken.

In other embodiments, the HCP reviews the data and determines if any changes to the prescription need to be made. In some embodiments, the data is reviewed on a health care professional interface app on a smart device, such as a computer, a smart phone, or a smart tablet. In certain embodiments, the HCP then sends the instructions for the change either to the disclosed respiratory devices, or to the user smart device, or both. The disclosed respiratory devices then alert the patient of the new instructions. Once the patient approves the instructions, the disclosed respiratory devices change the medication cocktail accordingly.

The instructions from the HCP may include increase or decrease of the medications' doses, changes to the combination of medications, increase or decrease the frequency of treatment with nebulized drug or hot steam, increase or decrease the session duration, contact the HCP immediately, go to an urgent care facility, or alert the emergency medical services. In some embodiments, the HCP instructs the disclosed respiratory devices to automatedly alert EMS either by a phone call with a recorded message, or by a text or other type of electronic message. In these embodiments, if the user has become incapacitated, then the HCP can summon immediate help by the push of the button, without wasting time to obtain the user's location to relay to the EMS.

An advantage of the presently disclosed devices 100 is that they are always ready to be used. Even when they are not being operated they are in stand-by mode where the device can be made operative in a matter of a few short seconds. Therefore, the present devices 100 are particularly suited to be used in emergency situations, where by just a single press on the start button the appropriate medication cocktail is delivered to the user.

The various aspects of the disclosed respiratory devices are now discussed with a view to the accompanying drawings, in which like numbers refer to like parts.

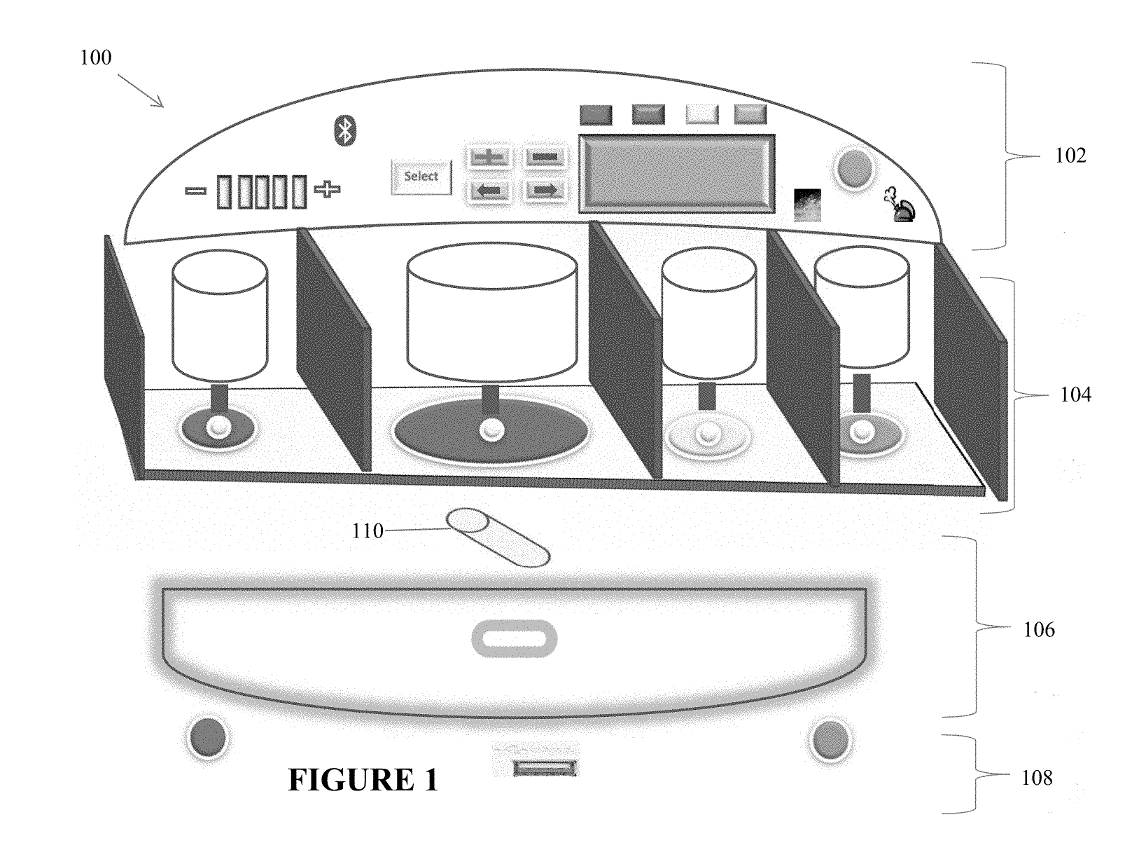

FIG. 1 illustrates an embodiment of the disclosed respiratory devices. The respiratory device 100 comprises four components: a control unit 102, a filling unit 104, a generation and output unit 106, and an input unit 108. In some embodiments, the device 100 comprises additional units. In other embodiments, some of the units are combined together. An output stick 110 is provided that delivers the output of the generation and output unit 106 to the user. Thus, the aforementioned units are not to be construed as physically separate components, but rather as one or more components that allow the function of the unit as described herein.

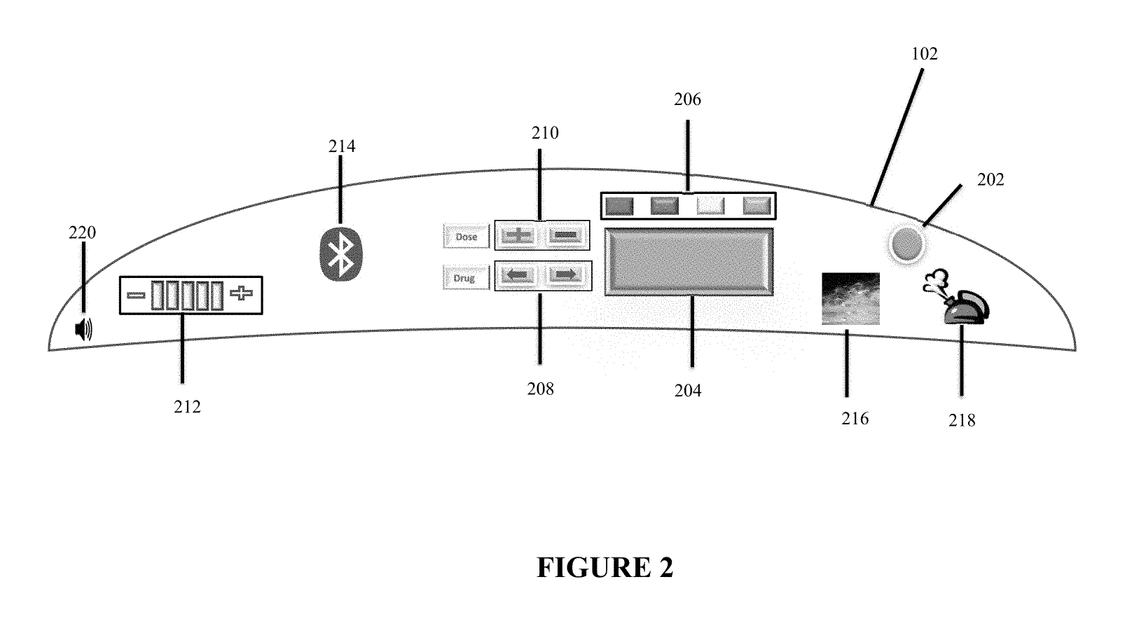

FIG. 2 illustrates an embodiment of the disclosed control unit 102. A power button 202 is provided. Activating the power button 202 turns the device 100 on, while deactivating the power button 202 turns the device 100 off. In some embodiments, the power button 202 also acts as a start button. The start button initiates the nebulization of the medication or steam generation, depending on the selected function. In other embodiments, the start button is a separate button.

Throughout the present disclosure, the word “button” is interchangeable with “switch” and denotes any device that can turn the flow of electricity on or off.

In some embodiments, the power button 202 is a toggle switch that alternates through a series of options. The following scenario is offered as an example of how various functions can be chosen. One press of the power button 202 powers the device 100 on and enables it to generate nebulized particles from the medications chambers 302 (see below) (nebulization mode). Two presses, either immediately consecutively or about 2-3 seconds apart switches the function of the device 100 to generate steam from a water tank, described below (steam mode). By long pressing, e.g., 2-3 seconds, on the power button 202 will allow you to the settings panel (see below) is accessed. In some embodiments, the user can choose only the nebulization mode or the steam mode, but not both. In certain embodiments, when it is clinically beneficial to have the nebulized medication delivered along with steam, the user can choose both nebulization and steam modes simultaneously.

In some embodiments, the control unit 102 further comprises a display monitor 204. The monitor 204 displays various numbers, messages, alerts, etc. In some embodiments, the monitor 204 is a liquid crystal display (LCD) monitor. Other types of displays, such as a dot matrix display, cathode ray tube, and the like, or display types developed in the future, are contemplated. In some embodiments, the display is well-illuminated, with clear and high resolutions figures and letters displayed in large font. It is contemplated that a significant number of the users of the device 100 will be very ill and/or elderly patients who may have difficulty reading digital displays. The monitor 204 is especially configured with such limitations in mind.

In some embodiments, the control unit 102 further comprises a set of chamber lights 206. Each chamber light 206 corresponds to one of the chambers 302,304 (discussed below). When the light 206 for a particular chamber 302,304 is on, then that particular chamber 302,304 is on active mode. Any value that is displayed on the monitor 204 relates to the particular chamber 302,304. Thus, for example, if the dosage is adjusted, then the dosage for the medication in the chamber 302 whose light 206 is illuminated is being adjusted. By way of example only, in some embodiments, each particular chamber 302,304 is color-coded. For example, a red chamber holds Drug A, while a yellow chamber holds Drug B. During the operation, a lit red light 206 indicates that the red chamber 302 settings are being adjusted, while a lit yellow light 206 is lit indicates that the red chamber 302 settings are being adjusted.

The embodiment shown in FIG. 2 shows four chamber lights 206. The ordinary artisan recognizes that there could be any number of chamber lights 206. In practice, the quantity of lights 206 should correspond to the quantity of chambers 302,304. However, in some embodiments, the device 100 is configured to accommodate 5, 7, 8, 10, or more chambers 302,304, but not all of which are placed in the device 100 because the user only requires two or three medications. In that case, the control unit 102 exhibits more chamber lights 206 than there are chambers 302,304.

In some embodiments, the chamber lights 206 are color coded, such that each light has a different color. In certain of these embodiments, the chambers 302,304 are also color coded and the color of the chamber 302,304 matches the color of the respective chamber light 206.

In some embodiments, the control unit 102 further comprises at least one set of navigation buttons 208. In some embodiments, there are at least two buttons per each set of navigation buttons 208. One button increases a value while another button decreases a value. In other functions, one button moves the cursor to the right, while another button moves the cursor to the left. In still other functions, button moves the cursor up, while another button moves the cursor down. In these embodiments, another button (not shown) allows the user to toggle through the various options that require adjustment.

In other embodiments, the control unit 102 comprises a plurality of sets of navigation buttons. For example, in the embodiment shown in FIG. 2, the control unit 102 comprises a set of navigation buttons 208 and a second set of navigation buttons 210. In these embodiments, the functions of the navigation buttons are distributed between the plurality of the sets of navigation buttons. In the example of FIG. 2, one set of navigation buttons 208 is used to select the drug whose dose is to be adjusted, while the second set of navigation buttons 210 is used to increase or decrease the dose. The ordinary artisan recognizes other functions, such as settings, functions, etc., can be adjusted using the illustrated navigation buttons 208,210 or any other set of, or individual, navigation buttons.

The following example is provided for illustration only and is not to be considered as limiting the scope of the accompanying claims in any way. The navigation buttons 208 are used to move between, or select, prompts and functions that are displayed on the monitor 204. The buttons 208 can also be used to move between the chambers 302,304 to adjust the dose of the drug whose chamber 302 is selected or the amount of distilled water used from chamber 304 to mix with the drug (i.e., change the concentration of the drug that is introduced into the nebulizer. Once a chamber 302,304 is selected, the chamber light 206 corresponding with that chamber 302,304 is illuminated. The user now knows that all further modifications will be made to the chamber 302,304 whose light is illuminated.

The user then adjusts the selected variable using the navigation buttons 210. For example, Chamber 1 contains salbutamol. To adjust its dose from 0.1 mL to 0.5 mL, Chamber 1 is selected using the buttons 208. Dose setting for Chamber 1 is also selected by using the buttons 208. The dose is then adjusted upwards using the buttons 210. In some embodiments, the dose changes in 0.1 mL increments, while in other embodiments, the increments are smaller, such as 0.01 or 0.05 mL, and in still other embodiments the increments are greater, for example 0.5 or 1.0 mL. In some embodiments, the HCP can determine what the dose adjustment increment for a particular chamber 302,304 should be. This is determined by the characteristics of the particular drug and its approved dosing regimen.

In some embodiments, the control unit 102 further comprises a nebulization rate indicator 212. In these embodiments, the indicator 212 is used to specify the length of the nebulization period. Based on the physiological data that the HCP has received, the HCP can determine whether the user requires the medication to be administered as rapidly as possible, or whether the medication should be administered over several minutes. Adjusting the nebulization rate allows for the adjusting of the administration time. If the nebulization rate is increased, the time needed to complete the administration of the dose becomes shorter, and vice versa.

In some embodiments, the device 100 is configured to connect wirelessly with other devices using Bluetooth® technology. In some of these embodiments, a light indicator 214 is provided to show when a connection is successfully made. In some embodiments, the light indicator 214 is also a button that can be depressed to activate the Bluetooth® pairing function of the device 100.

In some embodiments, the control unit 102 comprises indicators that show the user whether the nebulization or the steam mode is selected. In some embodiments, a notice on the monitor 204 (e.g., the words “Nebulization” and “Steam”) appears to indicate the status. In other embodiments, a separate display shows which mode is selected. In the embodiment shown in FIG. 2, a display light 216 is illuminated when the nebulization mode is selected, and a different display light 218 is illuminated when the steam mode is selected.

In some embodiments, the control unit 102 comprises a speaker 220. In some embodiments, the speaker 220 provides audible alerts for the user or a caregiver. In other embodiments, when the user has difficulty reading the messages displayed on the monitor 204, the messages are transmitted in spoken word through the speaker 220. In some embodiments, the user chooses whether some or all the information on the monitor 204 is to be vocalized. If a subset of the messages is to be vocalized, then the user can choose the members of the subset. In some embodiments, the selection is made through the use of the navigation buttons 208,210 and followed on the monitor 204.

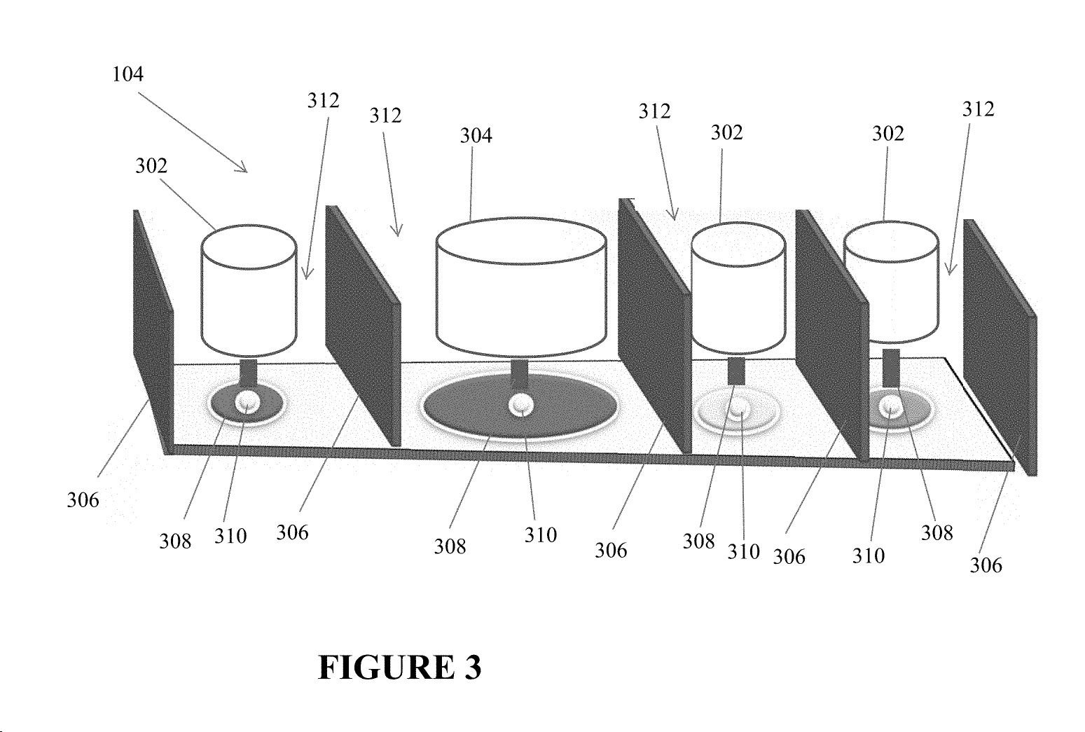

FIG. 3 illustrates an embodiment of the filling unit 104. The filling unit 104 comprises a plurality of medication chambers 302 and at least one distilled water chamber 304. In some embodiments, each chamber 302,304 is placed in its own separate housing 312, or chamber room, that is defined by walls 306 that separate the housing 312 from the adjoining housings 312, and by walls (not shown) in the front and back of the housing 312. In some embodiments, the entire filling unit 104 is pulled out of the device 100 to fill the chambers 302,304 with the appropriate medications or water and then the filling unit 104 is replaced in the device 100. In other embodiments, each chamber 302,304 is removed individually from its respective housing 312 in the filling unit 104 of the device 100 to be filled with medication or water. Each chamber is then replaced in its corresponding housing 312. In some of the embodiments where the chambers 302,304 are color-coded, the housings 312 are also color-coded and the color of the housing 312 corresponds to the color of the chambers 302,304.

The embodiment shown in FIG. 3 comprises three medication chambers 302 and one distilled water chamber 304. Consequently, the illustrated embodiment depicts a device that is useful for delivering at most three medications at a time to the patient. The ordinary artisan recognizes that other embodiments are directed to those having more, or less, than three medication chambers 302.

In some embodiments chamber 304 contains distilled water. The water is used to dilute the medication(s) before their use in the nebulizer. In some embodiments the patient is administered a combination of several drugs. The water in the chamber 304 is used to make a solution out of the one or a mixture of medications, thereby diluting the medication. A dilute solution of a medication results in minimizing the adverse events associated with the medication. Because the amount of medication being administered is not changed, the dilute solution results in better pharmacokinetic profile, i.e., a lower Cmax but the same AUC as compared to the administration of the concentrated drug. In some embodiments, the dilution step is employed while the device 100 is used in the nebulization mode.

While the above discussion was presented in terms of distilled water being the carrier, other carriers, i.e., physiologically acceptable solvents that can be volatilized, can also be used in lieu of water if the HCP prescribes the use of such carriers.

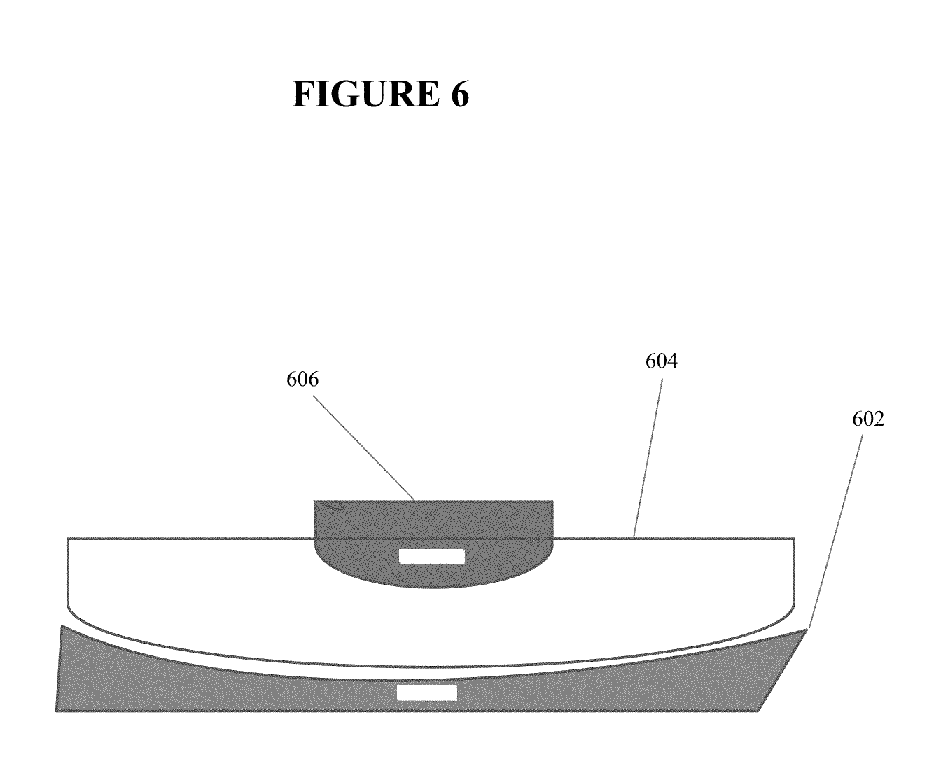

In some embodiments, each chamber 302,304 is connected by an outlet tube 308 to an opening 310. The administered material from chamber 302,304 is dispensed from the chamber 302,304 and into the opening 310 from where it is delivered through a conduit (not shown) to a medication collector (606, FIG. 6) in the generation and output unit 106 to form the prescribed medication cocktail. The cocktail is then nebulized and delivered via output stick 110 to the user.

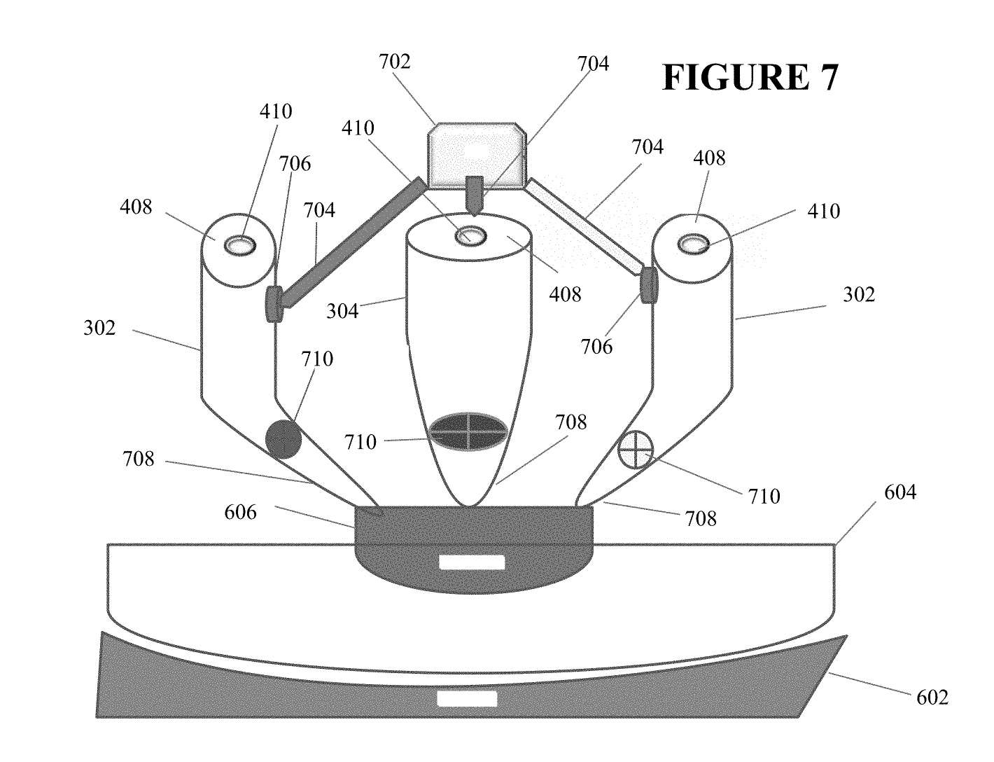

In some embodiments, the outlet tube 308 is a part of the chamber 302,304 while in other embodiments, the outlet tube 308 is a separate piece from the chamber 302,304. As described in detail below, each chamber 302,304 is connected by a conduit 708 (FIG. 7) to the generation and output unit 106, where a medication or a medication cocktail is ultrasonically nebulized to be delivered to the user.

In some embodiments, each chamber 302 has sufficient volume to hold enough medication to be used for a period of five or more days. In some embodiments, each chamber 302 has a volume of between about 1 to about 100 mL, or between about 5-75 mL, or between about 10-50 mL, or between about 20-40 mL. In some embodiments, each chamber 302 has a volume of about 30 mL. Thus, for example, if the medication in a chamber 302 is a salbutamol, budesonide, or ipratropium bromide (Atrovent®) solution, the chamber volume of 30 mL will provide a dose of 1 mL of the drug solution up to six times a day for a minimum 5 days.

In some embodiment, chamber 304 has sufficient volume to hold enough distilled water to be used for medication dilution for a period of five or more days. In some embodiments, chamber 304 has a volume of about 150 mL.

Throughout the present disclosure the term “about” a certain value means that a range of value ±25%, preferably about ±10%, and more preferably a range of value ±5%, is contemplated. Thus, for example, having a volume of about 10 mL includes volume being between 7.5 mL and 12.5 mL, and preferably between 9 mL and 11 mL, and more preferably between 9.5 mL and 10.5 mL. Furthermore, when about a range is given, it is understood that the word “about” qualifies both termini of the range. Thus, for example “about 7-10” means “about 7 to about 10.”

In the discussion herein reference is made to an “administrable material” in a chamber 302,304. In the present context, “administrable material” refers to a medication in a chamber 302, or water in a chamber 304.

In some embodiments, each chamber 302,304 comprises a sensor that obtains information regarding the level of administrable material (medication or water) in the chamber 302,304 and causes the level to be displayed on the monitor 204. In certain embodiments, if the administrable material levels fall below a certain pre-determined threshold level, then the sensor causes and audible sound to be emitted to alert the user.

In some embodiments, the device 100 is used on a permanent basis by the user. In other embodiments, an HCP provides the user with a device 100 in some acute or chronic settings for the user to use for a period of days or a few weeks. After the prescribed period of use is over, the user returns the device 100 to the HCP, who will in turn provide it to another user in need thereof after the device 100 is thoroughly cleaned and disinfected. In either case, there is a need to refill the chambers 302,304 with the proper medication frequently. The refilling is done either by the user, or a caregiver, when the user is a permanent user, or by the HCP or their staff when the HCP provides the device 100 to the user.

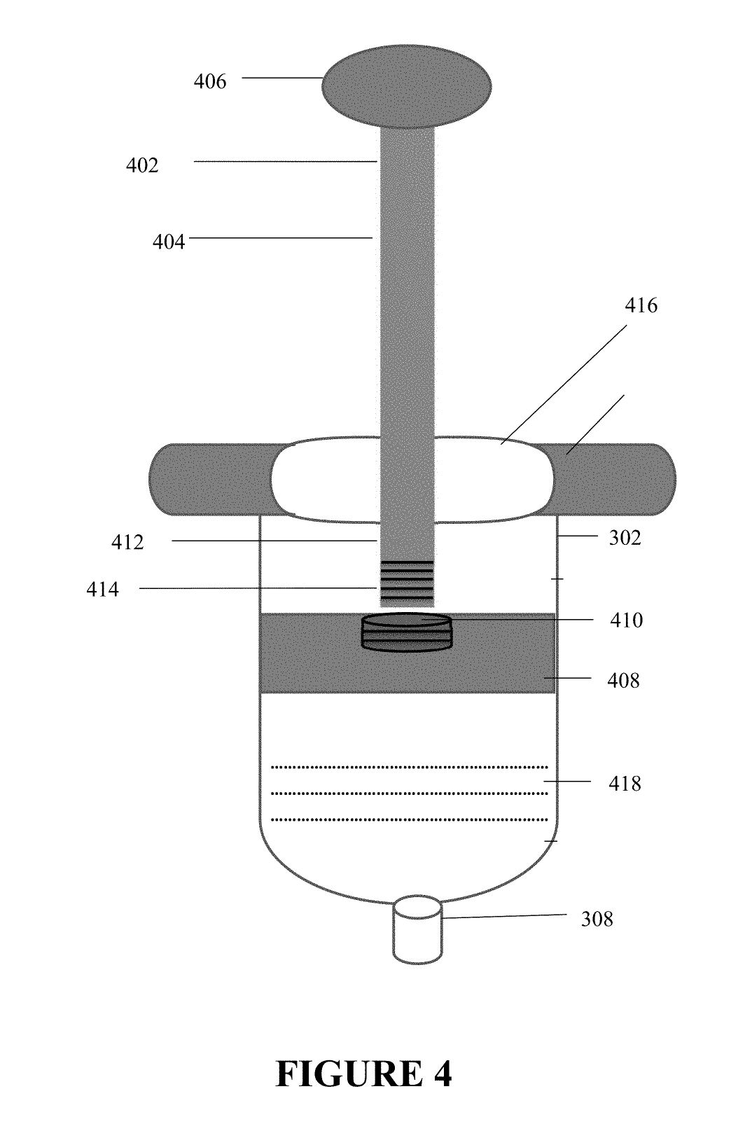

Accordingly, there is provided a mechanism to fill each chamber 302,304 with the corresponding medication. FIG. 4 illustrates an embodiment of the disclosed filling mechanism. The chamber 302 is separated from the filling unit 104 of the device 100. Each chamber 302 comprises a movable seal 408, which in turn comprises a joint 410. A plunger 402 is provided, which comprises a shaft 404 and a handle 406 at its proximal end. The distal end 412 of shaft 404 terminates in a plunger joint 414.

To fill a chamber 302,304, the distal end 412 of the plunger 402 is inserted through an opening in the upper end 416 of the chamber 302,304. The plunger joint 414 is then connected with the chamber joint 410. The connection of the two joints can be through any known connection means. For example, the two joints can be connected by threading the shaft joint 414 into the chamber joint 410, or connecting them through a friction lock mechanism. The plunger 402 is then pressed all the way down by pressing on the handle 406. When the seal 408 has reached the bottom, the outlet tube 308 is connected to a medication source. When the plunger 402 is pulled back by pulling on the handle 406, the seal 408 moves up, which causes a vacuum to be generated within the chamber 302,304, which in turn causes the medication to be sucked into the chamber 302,304. Fill lines 418 show the volume of medication within the chamber 302,304. Once sufficient administrable material has been added to the chamber administrable material, the plunger 402 movement is stopped, the plunger 402 is separated from the seal 408 and the chamber administrable material are returned to the filling unit 104, where the outlet 308 once again is connected with the opening 310.

In some embodiments, both the plunger 402 and the chamber 302,304 are made of materials that are easily cleaned and do not lend themselves to the accumulation of bacteria, mold, or other noxious organisms. In some embodiments, one or more of the chambers 302 are opaque to protect any light sensitive medications contained therein.

The ordinary artisan recognizes that the above is just one example of the mechanism by which chambers 302,304 can be filled. Other mechanisms are known to the skilled artisan. For example, in some embodiments the plunger movement is automated and is controlled by an electrical motor. In some of these embodiments, the plunger permanently stays attached to the seal 408. Conventional means of filling the chambers 302,304 are also contemplated. For example, a syringe can be used to obtain the proper amount of a material and then the syringe is emptied into the chamber 302,304.

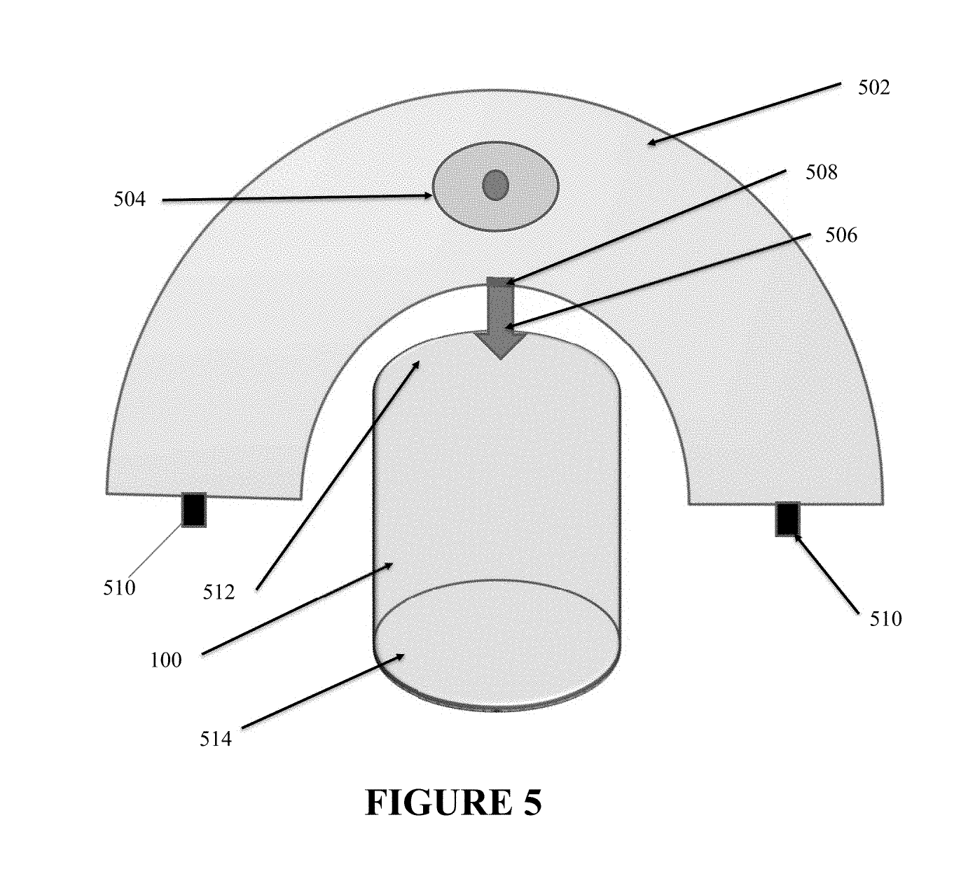

In some embodiments, the device 100 further comprises a water tank. FIG. 5 illustrates an embodiment of the water tank 502. The water tank 502 is connected to the device 100 when it is going to be used to produce steam. In some embodiments, the water tank 502 is removable. In certain of these embodiments, the water tank 502 is attached to the back of the device 100. In some of these embodiments, the water tank 502 is connected to the input unit 108. In some embodiments, the tank capacity is under 10 L, or under 8 L. In certain embodiments, the tank capacity is 5 L. In other embodiments, the tank capacity is less than 5 L, for example 4 L. In some embodiments, the tank comprises a sensor that alerts the user, and/or shuts off the steam generator, if the water level has fallen below a threshold level.

In some embodiments, the water tank 502 comprises a filling cap 504 through which the water tank 502 is filled with water, such as sterilized, deionized, steam-condensed, or filtered water.

In some embodiments, the water tank 502 is connected to the back of the device 100 by a conduit 506. In certain embodiments, the conduit 506 is located at the bottom of the water tank 502, whereas in other embodiments, the conduit 506 is located at the side of the water tank 502. In some embodiments, the conduit 506 has a one-way valve 508 that allows the water to pass from the water tank 502 to the unit 100 only, but not in the reverse direction. Accordingly, once the water tank 502 is connected to the device 100, water will pass from the water tank 502 to a heat generator (not shown) in the generation and output unit 106, where the heat generator heats up the water to boiling to generate steam. The steam is then delivered to the user via the output stick 110. The duration of hot steam inhalation session is determined through the control unit 102.

Thus, as can be seen, in some embodiments the output stick 110 is configured to deliver nebulized product, while in other embodiments, the output stick 110 is configured to deliver steam. In still other embodiments, such as the one contemplated by the illustrated embodiments, the output stick 110 delivers the nebulized product while operating in nebulization mode, and delivers steam while operating in steam mode.

In some embodiments, the water tank 502 comprises one or more connector(s) 510 that secure the water tank 502 to the device 100. In some embodiments, the water tank 502 comprises four connectors 510: two connectors 510 connects the water tank 502 to the lower end 512 of the device 100; and another two connectors 510 connects the water tank 502 to the upper end 514 of the device 100.

In some embodiments, the generation unit 106 comprises an ultrasound generator 602, as shown in FIG. 6. Adjacent to the ultrasound generator 602 is a water sink 604. The water sink 604 is in vibrational communication with the ultrasound generator 602. In other words, when the ultrasound generator generates an ultrasonic wave, the wave causes motional disturbances in the water of the water sink 604. In some embodiments, the water sink 604 is located above the ultrasonic generator 602. In some embodiments, the water sink 604 is sealed so that no water spills once the vibrations due to the ultrasound waves begin.

In some embodiments, the water sink 604 contains enough water to partially submerge the medication collector 606 within the water of the water sink 604. In some embodiments, between about one-fifth to about four-fifths of the medication collector 606 is submerged in the water. In some embodiments, between about one-fourth to about three-fourths of the medication collector 606 is submerged in the water. In some embodiments, between about one-third to about two-thirds of the medication collector 606 is submerged in the water. In some embodiments, about one-half of the medication collector 606 is submerged in the water.

As described above, medications that are dispensed from chambers 302,304 ultimately collect in the medication collector 606 to be nebulized. Once the medication or the cocktail of medications is completely collected, the ultrasound generator 602 generates an ultrasound wave that causes the medication or the cocktail of medications within the medication collector 606 to become nebulized. In certain of these embodiments, the ultrasonic generator generates a sound wave having a frequency of about 100 KHz. This frequency produces nebulized particles of size 1-5-micron, which is the proper particle size for a successful delivery to medium and small airways as well as the alveoli. Delivering the medication down to the alveoli makes the nebulization treatment more effective and more efficient compared to delivering the nebulized particles to the small and medium size airways only.

Accordingly, the generator and output unit 106 comprises the ultrasonic generator 602 for nebulization production, the heat generator for steam production, the sealed water sink 604, the medication collector 606, and the output stick 110 through which the nebulized product and/or steam are delivered to the user.

The input unit 108 comprises a plurality of USB ports that allow for other devices or flash memory drives to be connected to the device 100 for data transfer, either from the device 100 to the accessory or vice versa. In certain embodiments, other accessories, such as a pulse oximeter, an expiratory peak flowmeter, other physiological function sensors, and the water tank, communicate with the device 100 through the input unit 108.

FIG. 7 is a schematic showing the mechanism of the dispensation of administrable materials in the medication collector 606. In some embodiments, according to the HCP prescription the dose for each administrable material in each chamber 302,304 is set by using buttons in the control unit 102. When a dose is to be administered to the user, a pump 702 (or actuator) generates pressurized air that is sent through air pressure lines 704 to an air inlet 706. (In FIG. 7 for purposes of clarity the air inlet 706 of chamber 304 is not shown.) Each air inlet 706 is in pneumatic communication with the seal 408 of its respective chamber 302,304. Therefore, when the pressurized air hits the air inlet 706, the air pressure is transferred to the seal 408. The air pressure pushes the seal 408 down to eject an exact dose in volume of the administrable material from the outlet 308 of the chamber 302,304. Each outlet 308 is connected by a conduit 708 to the medication collector 606. In the absence of air pressure, or when the air pressure is not what it is set to be, a pressure valve 710 prevents any administrable material from inadvertently entering the conduit 708. In some embodiments, the conduit 708 is vibrated to ensure all the administrable material is delivered from the conduit 708 to the medication collector 606.

In some embodiments, the device 100 comprises a single conduit 708 that connects to each chamber 302,304 individually to deliver its administrable material to the medication collector 606. In other embodiments, each chamber 302,304 has its own conduit 708 which is separate and distinct from the conduits 708 of other chambers 302.

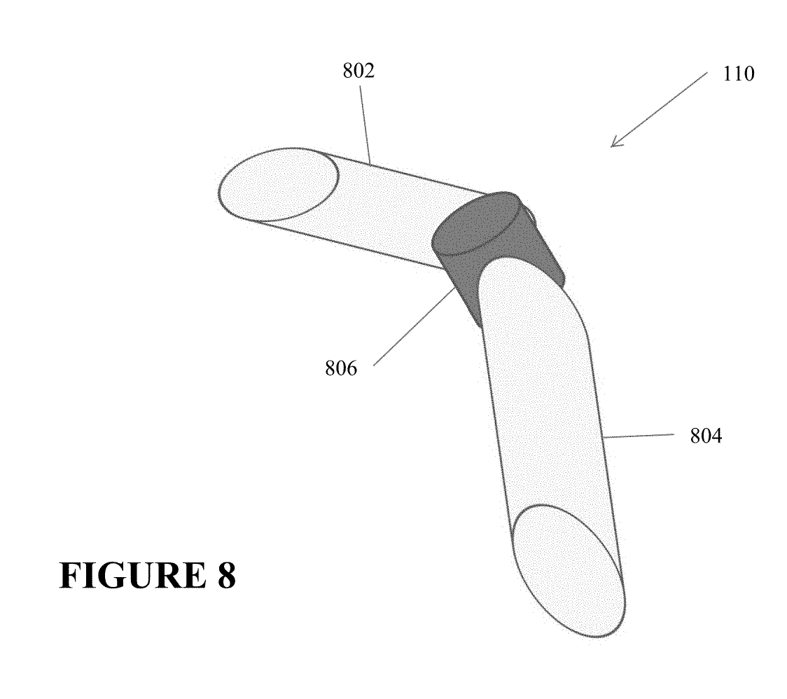

As stated above, in some embodiments, the device 100 comprises an output stick. FIG. 8 illustrates an embodiment of the disclosed output stick 110. The output stick 110 comprises a proximal section 804 and a distal section 806. The proximal section 804 connects to the device 100, whereas the distal section 806 connects to the user's respiration line. In some embodiments, each of the sections 804,806 is tubular having a generally circular or oval cross section. The two sections 804,806 are connected by a connector 808. In some embodiments, the connector 808 has rotating components that allow for the angle between the sections 804,806 to vary when needed. In some embodiments, the angle varies between 0°, i.e., when the two sections 804 and 806 are adjacent to each other, and 180°, i.e., when the two sections 804 and 806 form a straight line. In other embodiments, the connector 808 is a swivel which allows section 804 to swivel with respect to section 806, and vice versa.

In some embodiments, the output stick 110 is made of materials, such as plastics, that are easily cleanable. The output stick 110 can be cleaned with usual disinfecting and cleaning agents used for medical devices or by autoclave.

What is claimed is:

1. A respiratory device comprising at least four components selected from a control unit 102, a filling unit 104, a generation and output unit 106, and an input unit 108, wherein the respiratory device comprises a nebulizer mode and a steam mode of operation,
wherein the control unit 102 comprises at least one of:
a) a power button 202, which is a toggle switch that alternates through a series of options;
b) a display monitor 204;
c) a set of chamber lights 206;
d) at least one set of navigation buttons 208;
e) a first set of navigation buttons 208 and a second set of navigation buttons 210, wherein the first set of navigation buttons 208 is used to select a drug whose dose is to be adjusted, while the second set of navigation buttons 210 is used to increase or decrease the dose of the drug;
e) a nebulization rate indicator 212;
f) at least one indicator to show whether the nebulization or the steam mode is selected; and
g) a speaker 220.

2. The device of claim 1, wherein the nebulization rate indicator 212 allows a user to specify a length of a nebulization period.

3. The device of claim 1, wherein the filling unit 104 comprises a plurality of administrable material chambers 302,304 for one or more administrable material.

4. The device of claim 3, wherein the filling unit 104 comprises a plurality of medication chambers 302 at least one carrier chamber 304.

5. The device of claim 3, wherein each chamber light 206 corresponds to one of the administrable material chambers 302,304.

6. The device of claim 3, wherein each chamber 302,304 is connected by an outlet tube 308 to an opening 310, and wherein a conduit 708 connects the opening 310 of each respective chamber 302,304 to the medications collector 606 in the generation and output unit 106.

7. The device of claim 1, further comprising a water tank in fluid communication with a heating unit within the generation and output unit 106, the tank comprises a sensor that alerts the user, and/or shuts off the steam generator, if the water level falls below a threshold level.

8. The device of claim 1, wherein the generation and output unit 106 comprises an ultrasonic generator 602, generating a sound wave.

9. The device off claim 1, wherein the input unit 108 comprises a plurality of USB ports.

10. The device of claim 9, wherein the electronic accessory is selected from a pulse oximeter, an expiratory peak flowmeter, other physiological function sensors, and a water tank.

11. The device of claim 1, wherein the respiratory device 100 communicates electronically with one or more devices that obtain physiological data from the user.

12. The device of claim 1, an output stick 110 that delivers the output of the generation and output unit 106 to the user.

13. The device of claim 4, wherein the administrable material chamber 302,304 comprises:
a hollow body;
a chamber outlet tube 308;
a moveable seal 408 comprising a seal joint 410; and
a sensor that obtains information regarding the level of fluid in the chamber 302,304.

14. The chamber of claim 14, wherein the chamber comprises fill lines 418 on the body of the chamber.

15. A plunger 402, comprising
a shaft 404;
a handle 406; and
a plunger joint 414 at the plunger distal end 412, wherein the plunger joint 414 connects securely with a seal joint 410 of a moveable seal 408 of a administrable material chamber 302,304.

16. A method of introducing medication in liquid form into a administrable material chamber 302,304, the method comprising:
obtaining a administrable material chamber 302,304 and a plunger 402;
inserting the plunger distal end 412 through an upper end 416 of the administrable material chamber 302,304;
connecting the plunger joint 414 with the seal joint 410;
placing the moveable seal 408 at the distal end of the administrable material chamber 302,304;
placing the chamber outlet tube 308 in fluid communication with the liquid medication;
pulling on the plunger 402 to move the moveable seal 408 towards the proximal end of the chamber 302; and
disconnecting the plunger 402 from the moveable seal 408 and removing the plunger 402 from the administrable material chamber 302,304.

17. A method of adjusting the parameters for the administration of two or more medications to a patient, wherein the patient is using a respiratory device of claim 1, the method comprising:
transmitting a set of physiological data regarding the patient to a health care provider via the internet;
receiving a set of instructions from the health care provider via the internet;
displaying the set of instructions to the patient and obtaining the patient's approval of the instructions; and
if applicable, adjusting the parameters for the administration of two or more medications to a patient.

18. The method of claim 19, wherein the parameters for the administration of two or more medications to a patient are selected from dose, duration, and frequency of the administration of each of the two or more medications.

19. The method of claim 19, wherein the set of physiological data regarding the patient are selected form the respiratory rate, respiratory volume, pulse rate, blood oxygen saturation limits, blood pressure, or body temperature.

20. The method of claim 19, wherein the set of instructions include: increase or decrease of the medications' doses, changes to the combination of medications, increase or decrease the frequency of treatment with nebulized drug or hot steam, increase or decrease the session duration, contact the HCP immediately, go to an urgent care facility, instruct the user to alert the emergency medical services, or automatedly alert the emergency medical services.
